(19) United States
(12) Patent Application Publication
(10) Pub. No.: US 2017/0252323 A1
(43) Pub. Date: Sep. 7, 2017

(54) 4-[2-(Dipropylamino)ethyl]-1,3-dihydro-2H-indol-2-one (Ropinirole) a New Inhibitor of Jack bean Urease Enzyme: An Example of Drug Repurposing

(71) Applicants: Jalaluddin Azam Jalal Khan, Jeddah (SA); Muhammad Iqbal Choudhary, Karachi (PK); Maryam Abdu Abdullah AL-Ghamdi, Jeddah (SA); Etimad Huwait, Jeddah (SA); Atia-tul Wahab, Karachi (PK); Sumaira Javaid, Karachi (PK)

(72) Inventors: Jalaluddin Azam Jalal Khan, Jeddah (SA); Muhammad Iqbal Choudhary, Karachi (PK); Maryam Abdu Abdullah AL-Ghamdi, Jeddah (SA); Etimad Huwait, Jeddah (SA); Atia-tul Wahab, Karachi (PK); Sumaira Javaid, Karachi (PK)

(21) Appl. No.: 15/061,280

(22) Filed: Mar. 4, 2016

(51) Int. Cl. A61K 31/404

(52) U.S. Cl. CPC A61K 31/404

(57) ABSTRACT

This invention provides that Ropinirole (4-[2-(Dipropylamino)ethyl]-1,3-dihydro-2H-indol-2-one) possess potent urease inhibitory potential (in vitro) with 97.3% inhibition and IC₅₀=11.7+0.46 μM, when compared to the standard inhibitors i.e. acetohydroxamic acid (IC₅₀=41.5+1.50 μM), and thiourea (IC₅₀=21.0+0.50 μM).

### BACKGROUND OF THE INVENTION

The development and approval of new drugs is a difficult, costly and time consuming task with a high rate of failure. Drug repurposing is one of the most effective tools to achieve efficiency in the process of drug discovery and development. “Drug repurposing” or “therapeutic switching” is an efficient strategy for identifying new therapeutic uses of existing drugs. In the last few decades, drug repositioning has gained major scientific interest. It has the potential to overcome initial bottlenecks in the drug development process.

The major advantage is that the pharmacokinetic, pharmacodynamic, and toxicity profiles of existing drugs have already been studied during clinical trials. Thus, these drugs could rapidly be translated into Phase II and III clinical studies for new indications, and the associated time and cost could be significantly reduced. Using these concepts, Biovista (Virginia, USA) repositioned “pirlindol” for the treatment of multiple sclerosis (MS). Pirlindol is a reversible inhibitor of monoamine oxidase A (RIMA), which was initially approved in Russia and the European Union for the treatment of chronic depression, affective and psychotic disorders. Biovista has filed patents for pirlindol (BVA-201) for the treatment of MS.

The drug screening project in this application is focused on identifying new drugs for different ailments. In the present studies, urease inhibitory assay was employed to evaluate the activity of 4-[2-(dipropylamino)ethyl]-1,3-dihydro-2H-indol-2-one (Ropinirole). Ropinirole, a dopamine agonist of the non-ergoline class of medication, is an approved FDA drug for the treatment of Parkinson's disease, extrapyramidal symptoms and restless legs syndrome (RLS). It is manufactured by GlaxoSmithKline (GSK), Cipla, Dr. Reddy's Laboratories, and Sun Pharmaceuticals.

Urease (EC 3.5.1.5.) is a nickel metallo enzyme (bi-nickel active site) that catalyses the hydrolysis of urea into carbon dioxide and ammonia. It is widely found in a variety of organisms, such as yeast, fungi, algae, bacteria and several higher plants. Urease plays an important role in seed germination and for the growth of microorganisms by breakdown of urea into ammonia. In plants and fungi, the urease enzyme exist as homopolymer while bacterial ureases, are heteropolymer (consist of two/three different subunits).

The inhibition of enzyme urease were extensively studied because of two main reasons (i) their role to protect soil from pH elevation and loss of nitrogen after use of urea fertilizer by controlling hydrolysis of urea in soil, and (ii) to discover effective therapy against bacterial urease (e.g. Helicobacter pylori, and Proteus mirabilis) that induce human pathogenic conditions, such as urinary stone formation, peptic ulcer, pyelonephritis, hepatic coma, and renal stones. Clinically hydroxamic acid, bismuth complexes, and imidazole classes are used to inhibit urease activity. However, these medicines are largely associated with numerous adverse effects, such as hydroxamic acid may cause hemolytic anemia, deep vein thrombosis. This created a demand for new inhibitors of urease enzyme in pharmaceutical research and development.

During our studies Jack bean (Canavalia ensiformis) urease was used for in-vitro biochemical evaluation of Ropinirole. The Jack bean urease shows identical active side residue with bacterial ureases. Therefore, it can serve as a primary screening model of urease for inhibitory studies because of the similarity in active site residues of all known origin of the ureases. The mechanistic studies were also carried out to determine the binding mechanism to urease.

### BRIEF SUMMARY OF THE INVENTION

Enzyme urease plays an important role in several pathologies, such as urolithiasis, urinary catheter encrustation, hepatic encephalopathy, peptic ulcers, and gastric cancers. Its inhibition, therefore, has a major therapeutic significance. For this purpose, we evaluated the urease inhibitory potential of 4-[2-(dipropylamino)ethyl]-1,3-dihydro-2H-indol-2-one (Ropinirole), i.e. a drug used for the treatment of Parkinson's disease and restless legs syndrome. This study was carried out by using Jack bean (Canavalia ensiformis) urease (EC 3.5.1.5) to evaluate the urease inhibitory activity of 4-[2-(dipropylamino)ethyl]-1,3-dihydro-2H-indol-2-one (Ropinirole). Furthermore, mechanistic studies were also carried out to determine the binding mechanism of this drug with urease enzyme.

### BRIEF DESCRIPTION OF THE DRAWING

FIG. 1 depicts the structure of 4-[2-(dipropylamino)ethyl]-1,3-dihydro-2H-indol-2-one (Ropinirole), evaluated for urease inhibitory activity.

FIG. 2A depicts the Lineweaver-Burk plot of rate of reaction (velocities) of Ropinirole verses reciprocal of substrate (urea) in the absence (▾), and in the presence of 5 μM (∇), 10 μM (▴), and 20 μM (Δ) of 4[2-(dipropylamino)ethyl]-1,3-dihydro-2H-indol-2-one (Ropinirole).

FIG. 2B depicts the secondary re-plot of Lineweaver-Burk plot for the determination of Ki, which is plotted between the slopes of each line on Lineweaver-Burk plot versus different concentrations of 4-[2-(dipropylamino)ethyl]-1,3-dihydro-2H-indol-2-one (Ropinirole).

FIG. 2C depicts the Dixon plot of rate of reaction of 4-[2-(dipropylamino)ethyl]-1,3-dihydro-2H-indol-2-one (Ropinirole) (velocities) versus its different concentrations. This plot indicate type of inhibition as a mixed-type against urease enzyme.

### DETAILED DESCRIPTION OF THE INVENTION:

Urease Inhibition Assay (Indophenol's Method):

Urease activity through indophenols method was measured by the production of ammonia, as described by Weatherburn with slight modifications. All reactions were performed in triplicate in a final reaction volume of 200 μL. Reaction mixtures consisting of 25 μL of Jack bean (Canavalia ensiformis) urease, 55 μL of buffer at pH 6.8, 100 mM of urea, and 5 μL of various concentrations of 4-[2-(dipropylamino)ethyl]-1,3-dihydro-2H-indol-2-one (Ropinirole) (i.e. from 0.5 to 0.00625 mM) were incubated at 30° C. for 15 min in 96-well plates. Subsequently 45 μL phenol reagents (1% w/v phenol and 0.005% w/v sodium nitroprussside), and 70 μL of alkali reagent (0.5% w/v NaOH and 0.1% w/v NaOCl) were added to each well. After 50 min, the increasing absorbance/optical density (OD) at 630 nm was measured by using a microplate reader (SpectraMax M5, Molecular Devices, Calif., USA). The percent inhibition was calculated from the formula given below:

% Inhibition=100−(OD test /OD control)×100.

Mechanistic Studies:

Mechanistic studies were carried out to determine the mechanism of inhibition of 4-[2-(dipropylamino)ethyl]-1,3-dihydro-2H-indol-2-one ( Ropinirole) of the enzyme urease. In brief, urease enzyme solution (1 U/200 μL) was incubated with different concentrations of Ropinirole for 15 min at 30° C. The reaction was then initiated by adding different concentrations of substrate (0.5-4.0 mM), then phenol and alkali reagent were added and change in absorbance was measured for 50 minutes at 630 nm on microtitre plate reader (SpectraMax M5, Molecular Devices, Calif., USA). The Lineweaver-Burk plot was used to determine the type of inhibition, while secondary replot of Lineweaver-Burk plot and Dixon plots were used for the determination and confirmation of dissociation constants (Ki).

Statistical Analysis:

The EZ-Fit enzyme kinetics program (Perrella Scientific Inc., Amherst, USA) was employed to calculate the IC₅₀ values. All graphs were plotted by using Grafit 7 (Erithacus Software Limited, UK). Values of the correlation coefficients, intercepts, slopes, and their standard errors were calculated by the linear regression analysis by using the same program. Each point in the constructed graphs represents the mean of the three experiments.

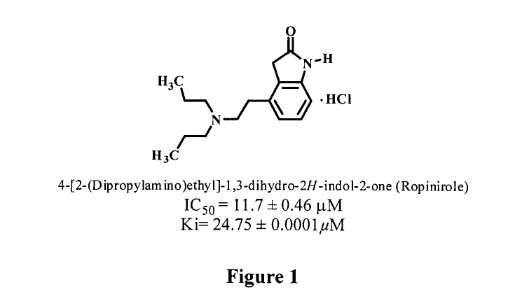

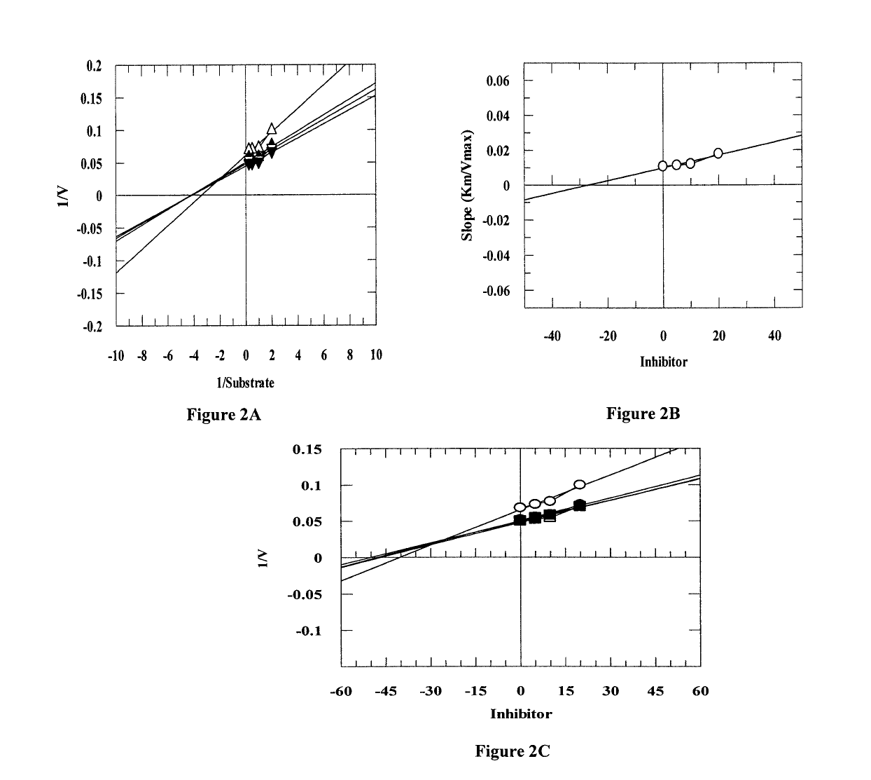

4-[2-(Dipropylamino)ethyl]-1,3-dihydro-2H-indol-2-one (Ropinirole) was found to be a potent inhibitor of urease enzyme with IC₅₀=11.7±0.46 μM (FIG. 1), when compared to standard inhibitors i.e. acetohydroxamic acid (IC₅₀=41.5±1.50 μM) and thiourea (IC₅₀=21.0±0.50 μM). As per literature survey, this is the first report describing the urease inhibitory activity of 4-[2-(Dipropylamino)ethyl]-1,3-dihydro-2H-indol-2-one (Ropinirole). Mechanistic studies revealed that it is a mixed-type of inhibitor of urease enzyme. This indicates that Ropinirole may interact with both the active and allosteric sites of the enzyme.

1-4. (canceled)

5. A urease enzyme inhibitor, 4-[2-(Dipropylamino) ethyl]-1,3-dihydro-2H-indol-2-one.

6. The urease enzyme inhibitor of claim 5, wherein said urease enzyme inhibitor is used to treat diseases caused by urease enzyme in humans and animals.
